(19) United States
(12) Patent Application Publication
(10) Pub. No.: US 2019/0298227 A1
(43) Pub. Date: Oct. 3, 2019

(54) TREMOR DETECTOR, STRESS ASSESSMENT SYSTEM INCLUDING THE SAME, AND METHOD OF ASSESSING STRESS

(71) Applicant: NEC CORPORATION, Tokyo (JP)

(72) Inventor: Yoshiki NAKASHIMA, Tokyo (JP)

(73) Assignee: NEC CORPORATION, Tokyo (JP)

(21) Appl. No.: 16/308,186

(22) PCT Filed: Jun. 5, 2017
(86) PCT No.: PCT/JP2017/020754

§ 371 Date: Dec. 7, 2018

(30) Foreign Application Priority Data
Jun. 8, 2016 (JP) 2016-114380

(51) Int. Cl. A61B 5/11; A61B 5/16

(52) U.S. Cl. CPC A61B 5/1101; A61B 5/6891; A61B 5/165

(57) ABSTRACT

A mental load cannot be appropriately evaluated when a tremor is detected by wearing an acceleration sensor on an arm and the like of a test subject; therefore, a tremor detector according to an exemplary aspect of the present invention includes pressure distribution detecting means for detecting pressures on a plurality of places in a plane and generating pressure information of information on pressure distribution in the plane; feature quantity extracting means for extracting a feature quantity from the pressure information; and tremor feature quantity extracting means for extracting a tremor-frequency feature quantity that is a frequency component of the feature quantity and includes a frequency component corresponding to a physiological tremor.

### TECHNICAL FIELD

The present invention relates to tremor detectors, stress assessment systems including the tremor detectors, and methods of assessing stress and, in particular, to a non-wearable tremor detector, a stress assessment system including the non-wearable tremor detector, and a method of assessing stress.

### BACKGROUND ART

In recent years, measures to cope with mental load (mental stress) of office-workers have become social challenges. It is known that the mental stress causes a tremor to fluctuate. The tremor is defined as unconscious mechanical vibrations in a human body site having an amplitude that is too minute to be apparent (approximately 10 micrometers (μm), for example). In particular, the tremor of an able-bodied person is referred to a physiological tremor.

Patent Literature 1 discloses an example of a fatigue inspection device and a fatigue evaluation method capable of measuring the fatigue of a person by using above-described vibration characteristics of the tremor. The related fatigue inspection device described in Patent Literature 1 includes a detection unit that detects vibration of a tremor, an analyzing unit that analyzes a spectrum of the vibration, and a display unit that displays data output from the analyzing unit. The detection unit is an acceleration sensor, and sends an output such as a voltage based on an acceleration to the analyzing unit.

The analyzing unit has a function of performing analog-to-digital (AD) conversion on an input, a function of acquiring a vibration spectrum by performing Fourier transform on obtained digital data, and a function of acquiring a spectrum content rate with respect to each band in a vibration spectrum.

The display unit displays data about the vibration spectrum obtained by the analyzing unit by means of a display or a printer.

In the related fatigue evaluation method described in Patent Literature 1, first, an acceleration sensor serving as the detection unit is attached to an arm of a person to be measured. Then the analyzing unit analyzes acceleration data obtained from the acceleration sensor. This provides a vibration spectrum and a spectrum content rate with respect to each band in total power. Here, in deriving the spectrum content rate with respect to each band, for an upper limb tremor, a frequency band of a high frequency component is set from 5 Hz to 50 Hz, and a frequency band of a low frequency component is set from 0.5 to 5 Hz. In such a spectrum of the upper limb tremor, when the components in the high frequency band are large, the fatigue is evaluated to be large in the cerebral system. On the other hand, when the components in the low frequency band are large, the fatigue is evaluated to be large in the spinal system.

It is said that, according to the related fatigue inspection device and the related fatigue evaluation method, the above-described configuration makes it possible to indicate a degree of fatigue quantitatively using a spectrum content rate and perform fatigue evaluation accurately.

The related technologies include technologies described in Patent Literature 2 and Patent Literature 3.

### CITATION LIST

### Patent Literature

[PTL 1] WO2002/094091

[PTL 2] Japanese Unexamined Patent Application Publication No. 2012-075708

[PTL 3] Japanese Unexamined Patent Application Publication No. H05-224771

### SUMMARY OF INVENTION

### Technical Problem

The above-mentioned related fatigue inspection device is configured to attach the acceleration sensor to an arm of a test subject in order to inspect a mental load (stress) of the test subject. In this case, wearing the acceleration sensor on a body is stressful to the test subject. The obtained acceleration data differ depending on the way that the acceleration sensor is attached. Reliable data can be obtained if the acceleration sensor is attached tightly; however, the stress on the test subject increases. Consequently, there is the problem that a mental load cannot be appropriately evaluated.

As described above, there has been the problem that a mental load cannot be appropriately evaluated when a tremor is detected by wearing an acceleration sensor on an arm and the like of a test subject.

The object of the present invention is to provide a tremor detector, a stress assessment system including the tremor detector, and a method of assessing stress that solve the above-mentioned problem that a mental load cannot be appropriately evaluated when a tremor is detected by wearing an acceleration sensor on an arm and the like of a test subject.

### Solution to Problem

A tremor detector according to an exemplary aspect of the present invention includes pressure distribution detecting means for detecting pressures on a plurality of places in a plane and generating pressure information of information on pressure distribution in the plane; feature quantity extracting means for extracting a feature quantity from the pressure information; and tremor feature quantity extracting means for extracting a tremor-frequency feature quantity that is a frequency component of the feature quantity and includes a frequency component corresponding to a physiological tremor.

A method of assessing stress according to an exemplary aspect of the present invention includes detecting pressures on a plurality of places in a plane, and generating pressure information of information on pressure distribution in the plane; extracting a feature quantity from the pressure information; extracting a tremor-frequency feature quantity that is a frequency component of the feature quantity and includes a frequency component corresponding to a physiological tremor; calculating a change rate of the tremor-frequency feature quantity with respect to time; and evaluating a mental load of a person exhibiting the physiological tremor based on the change rate.

### Advantageous Effects of Invention

According to a tremor detector, a stress assessment system including the tremor detector, and a method of assessing stress of the present invention, a mental load can be appropriately evaluated without placing a mental load on a test subject.

### BRIEF DESCRIPTION OF DRAWINGS

FIG. 1 is a block diagram illustrating a configuration of a tremor detector according to a first example embodiment of the present invention.

FIG. 2 is a schematic diagram schematically illustrating a configuration of a stress assessment system according to a second example embodiment of the present invention.

FIG. 3 is a block diagram illustrating a configuration of the stress assessment system according to the second example embodiment of the present invention.

FIG. 4 is a diagram illustrating results of stress evaluation obtained by using the stress assessment system according to the second example embodiment of the present invention, and illustrating the results obtained by Stroop test.

FIG. 5 is a diagram illustrating results of stress evaluation obtained by the stress assessment system according to the second example embodiment of the present invention, and illustrating the results when information task is assigned.

### EXAMPLE EMBODIMENT

Example embodiments of the present invention will be described below with reference to drawings.

### First Example Embodiment

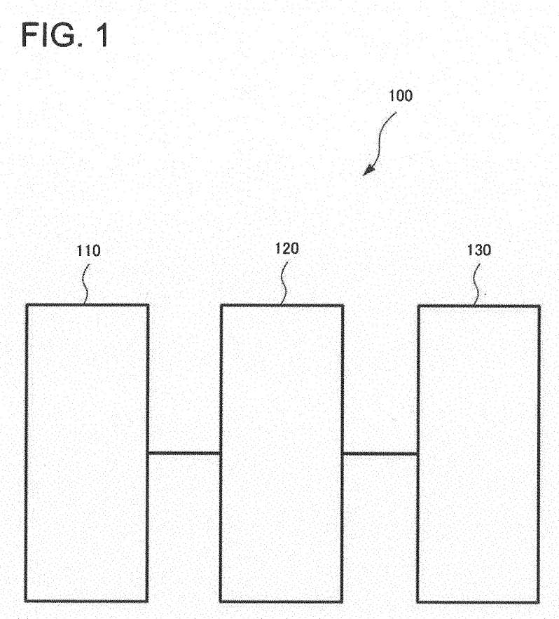

FIG. 1 is a block diagram illustrating a configuration of a tremor detector 100 according to a first example embodiment of the present invention. The tremor detector 100 includes a pressure distribution detecting means 110, a feature quantity extracting means 120, and a tremor feature quantity extracting means 130.

The pressure distribution detecting means 110 detects pressures on a plurality of places in a plane and generates pressure information of information on pressure distribution in the plane. As the pressure distribution detecting means 110, a sheet-like pressure distribution sensor can be typically used.

The feature quantity extracting means 120 extracts a feature quantity from the pressure information. The tremor feature quantity extracting means 130 extracts a tremor-frequency feature quantity that is a frequency component of the feature quantity and includes a frequency component corresponding to a physiological tremor. Here, the physiological tremor is defined as unconscious mechanical vibrations, in a human body site, having an amplitude that is too minute to be apparent.

The pressure distribution detecting means 110 can be placed in close contact with a test subject by weight of the test subject exhibiting a physiological tremor. Consequently, it is unnecessary that an acceleration sensor, as the above-mentioned related fatigue inspection device, should be worn on an arm and the like of a test subject in order to detect a tremor at a terminal of a body. As a result, including the pressure distribution detecting means 110 makes it possible to detect pressure information based on a physiological tremor of a body trunk without placing a burden on a test subject.

As described above, according to the tremor detector 100 in the present example embodiment, it is possible to evaluate a mental load appropriately without placing a mental load on a test subject.

Here, a feature quantity that the feature quantity extracting means 120 extracts from the pressure information can include at least one of central coordinates and a total sum of pressures in the pressure distribution.

### Second Example Embodiment

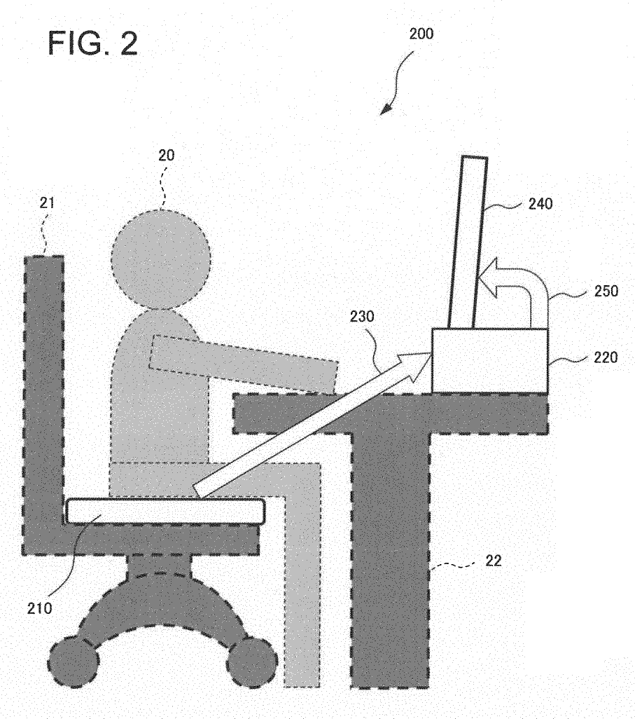

Next, a second example embodiment of the present invention will be described. FIG. 2 schematically illustrates a configuration of a stress assessment system 200 according to the second example embodiment of the present invention.

The stress assessment system 200 according to the present example embodiment includes a pressure distribution sensor (pressure distribution detecting means) 210, a stress evaluation apparatus 220, a pressure data transmission means 230, a stress-evaluation-result display (evaluation result display means) 240, and a stress-evaluation-result transmission means 250.

The pressure distribution sensor 210 is configured so that it will be placed on at least one of a seat and a back of a chair 21 for a person exhibiting a physiological tremor (test subject 20) to sit on, or under the test subject 20 who sits on the chair 21. FIG. 2 illustrates an example in which the pressure distribution sensor 210 is placed on the seat of the chair 21.

The pressure distribution sensor 210 has resolution for detecting an amplitude oscillating with acceleration of approximately 0.1 gal (Gal) and frequency of approximately 10 hertz (Hz). Here, 1 Gal=0.01 m/s²=1 cm/s². Specifically, for example, the pressure distribution sensor 210 can be configured that has at least 0.25-micrometer spatial resolution and at least 0.08-pascal pressure resolution, and operates at a sampling rate of at least 20 hertz.

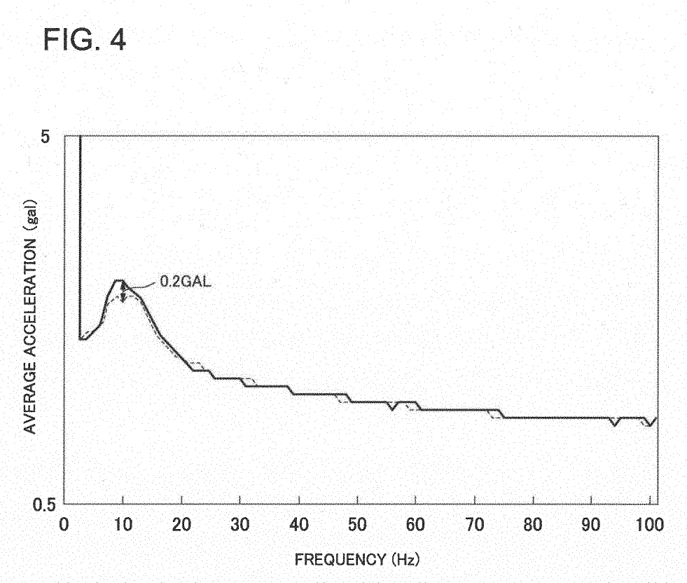

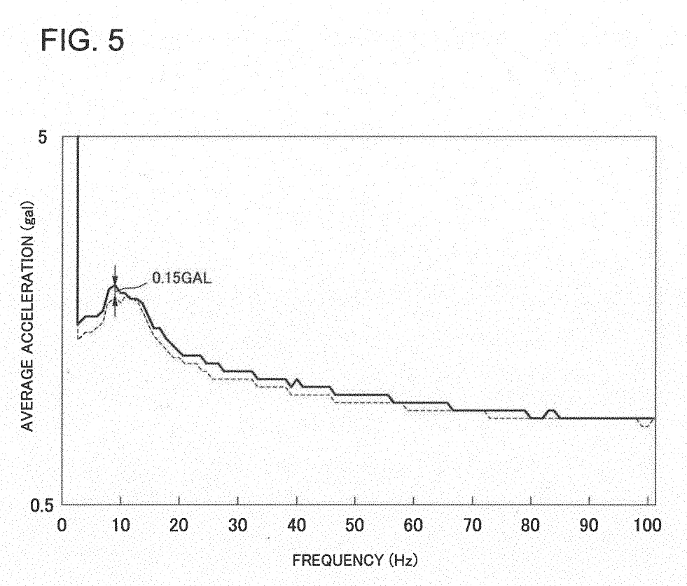

The above-mentioned accuracy of the pressure distribution sensor 210 has been unveiled from the experiments with the inventors, which will be described below using FIG. 4 and FIG. 5. That is to say, it has been unveiled from the experiments with the inventors that a difference of approximately 0.15 gal to approximately 0.2 gal in approximately 10 hertz (Hz) frequency component is caused in acceleration due to a physiological tremor of a body trunk of a test subject between the duration of stress being applied and the duration of a relaxed state. In order to detect the difference in the acceleration, the pressure distribution sensor 210 is configured to ensure the above-mentioned accuracy.

That is to say, in order to detect approximately 10 hertz (Hz) frequency component of the acceleration due to a physiological tremor, the pressure distribution sensor 210 is configured to operate at a sampling rate of at least 20 hertz (Hz) based on the sampling theorem. It is more preferable that the pressure distribution sensor 210 should be configured to operate at a sampling rate of 40 hertz (Hz). This is because operating at the sampling rate of 40 hertz (Hz) makes it possible to measure a frequency component up to 20 hertz and distinguish a peak at a frequency near to 8 to 10 hertz (Hz) as clearly distinguished from a frequency band before and after the peak.

When a physiological tremor horizontally vibrates at 10 hertz (Hz) with the acceleration of 0.1 gal (Gal) in a plane of the pressure distribution sensor 210, the maximum amplitude of the physiological tremor is approximately 0.25 micrometer (μm) on the assumption that the physiological tremor vibrates at simple harmonic motion. This value corresponds to the horizontal resolution to be required for the pressure distribution sensor 210, namely, the shortest distance between pressure detection cells.

When the pressure distribution sensor 210 is placed on the seat of the chair 21, the lower limit of weight of the test subject 20 can be 20 kilograms (kg), and the shape on the seat side of the pressure distribution sensor 210 can be a square, 50 centimeters (cm) on a side, in a typical example. In this case, when a vertical vibration with acceleration of 0.1 gal (Gal) occurs with a physiological tremor, the pressure applied to the pressure distribution sensor 210 becomes 0.08 pascal. This value corresponds to the pressure resolution required for each pressure detection cell constituting the pressure distribution sensor 210.

The pressure data transmission means 230 transmits pressure data (pressure information) from the pressure distribution sensor 210 to the stress evaluation apparatus 220. Here, the pressure data are data output from the pressure distribution sensor 210 and data of the pressure obtained by each pressure detection cell constituting the pressure distribution sensor 210. A signal processing process in a following stage makes it possible to obtain, from the pressure data, central coordinates in pressure distribution (positional data), frequency analytical data on variations in pressure at the central coordinates and in the entire pressure (frequency data), and the like. It makes no difference if the pressure data transmission means 230 is a wired means or a wireless means.

When the pressure data transmission means 230 is a wireless means, the test subject 20 can freely move the chair 21.

FIG. 2 illustrates the example in which the stress evaluation apparatus 220 is disposed on a table 22, to which the configuration is not limited. The stress evaluation apparatus 220 may also be attached to the chair 21. In this case, the test subject 20 can freely move the chair 21 even when the pressure data transmission means 230 is a wired means.

The stress evaluation apparatus 220 receives pressure data (pressure information) from the pressure distribution sensor 210 and assesses the stress of the test subject 20. The stress evaluation apparatus 220 can be configured by dedicated hardware. The configuration is not limited to this, and the stress evaluation apparatus 220 may be configured by an information processing system in which a central processing unit (CPU) executes a program stored in a memory. The information processing system constituting the stress evaluation apparatus 220 can be mounted in an information processing terminal and the like such as a desktop personal computer (PC), a notebook PC, and a smartphone.

The stress-evaluation-result display (evaluation result display means) 240 displays the evaluation results of a mental load (stress) obtained by the stress evaluation apparatus 220. When the stress evaluation apparatus 220 is configured by the information processing system mounted in the above-mentioned information processing terminal, an information display such as a display included in the information processing terminal can be suitably used as the stress-evaluation-result display 240. An information display apparatus and the like such as a display connected to the information processing terminal may be used as the stress-evaluation-result display 240.

The stress-evaluation-result transmission means 250 transmits the evaluation results of a mental load (stress) from the stress evaluation apparatus 220 to the stress-evaluation-result display 240. It makes no difference if the stress-evaluation-result transmission means 250 is a wired means or a wireless means. When the stress evaluation apparatus 220 is attached to the chair 21, it enables the test subject 20 to move the chair 21 freely that the stress-evaluation-result transmission means 250 is configured using a wireless means.

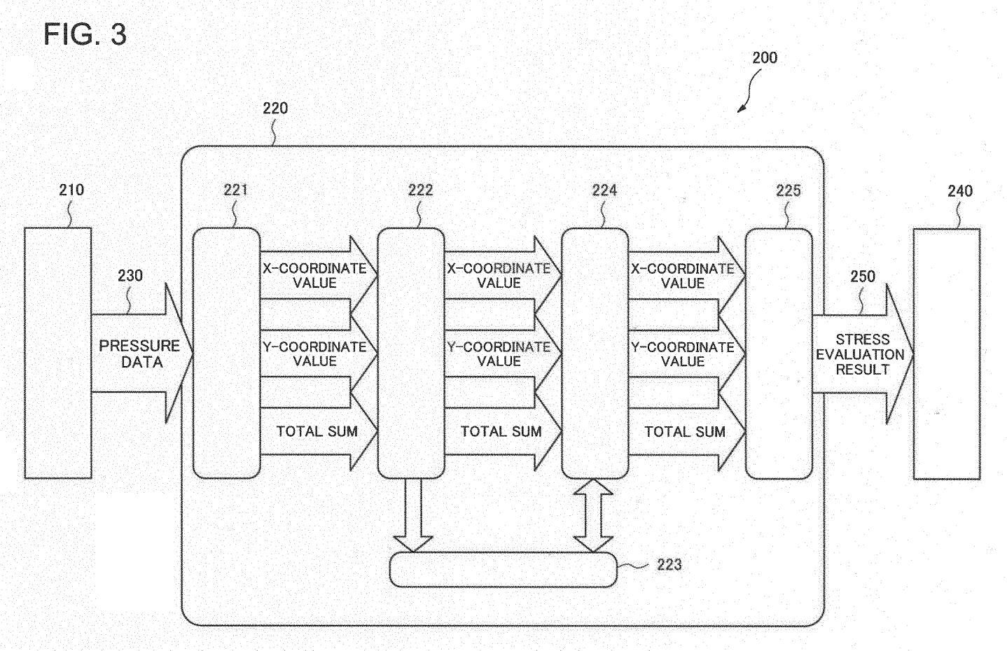

Next, the configuration and operations of the stress assessment system 200 according to the present example embodiment will be described in more detail. FIG. 3 is a block diagram illustrating a configuration of the stress assessment system 200 according to the present example embodiment.

As mentioned above, the stress assessment system 200 includes the pressure distribution sensor (pressure distribution detecting means) 210, the stress evaluation apparatus 220, the pressure data transmission means 230, the stress-evaluation-result display (evaluation result display means) 240, and the stress-evaluation-result transmission means 250.

The stress evaluation apparatus 220 receives pressure data from the pressure distribution sensor 210 through the pressure data transmission means 230, and evaluates the stress based on the pressure data. Then the stress evaluation apparatus 220 transmits evaluation results of the stress to the stress-evaluation-result display 240 through the stress-evaluation-result transmission means 250.

The stress evaluation apparatus 220 includes a feature quantity data computing part (feature quantity extracting means) 221 and a physiological tremor frequency data computing part (tremor feature quantity extracting means) 222. Here, a tremor detector is configured by the pressure distribution sensor (pressure distribution detecting means) 210, the feature quantity data computing part (feature quantity extracting means) 221, and the physiological tremor frequency data computing part (tremor feature quantity extracting means) 222. The stress evaluation apparatus 220 also includes a data storage part (feature quantity storage means) 223, a change rate data computing part (change rate calculation means) 224, and a stress evaluation computing part (load evaluation means) 225.

The feature quantity data computing part 221 extracts, from a physiological tremor, a feature quantity to evaluate stress based on the pressure data (pressure information) obtained from the pressure distribution sensor 210. The total sum of pressure values of respective pressure detection cells can be used as a feature quantity, for example. A physiological tremor vibrates not only in a vertical direction but also in an in-plane direction. This makes it possible to use, as a feature quantity, the central coordinates in the pressure distribution, namely, an X-coordinate value and a Y-coordinate value of a pressure center point. Specifically, for example, a longitudinal pressure distribution and a transverse pressure distribution are calculated using pressure values output from each pressure detection cell, and coordinates of a position at which the pressure value is equal to an average value in each direction can be used as a feature quantity.

The feature quantity data computing part 221 sends the X-coordinate value, the Y-coordinate value, and the total sum of pressure values, which have been calculated above, to the physiological tremor frequency data computing part 222.

The physiological tremor frequency data computing part 222 extracts a tremor-frequency feature quantity including a frequency component that is a frequency component of the feature quantity and corresponds to a physiological tremor. Here, the tremor-frequency feature quantity can include a frequency component at 10 hertz of the feature quantity. Specifically, for example, the physiological tremor frequency data computing part 222 can be configured to extract feature quantity data in the frequency band ranging from 8 hertz (Hz) to 10 hertz (Hz) by performing Fourier analysis processing on the received feature quantity data. In this case, a 10-second average value of the extracted feature quantity data may be calculated, for example.

The physiological tremor frequency data computing part 222 sends the tremor-frequency feature quantity to the change rate data computing part 224 and the data storage part 223.

The data storage part 223 stores the tremor-frequency feature quantity received from the physiological tremor frequency data computing part 222. The data storage part 223 can be configured to store previous data including only tremor-frequency feature quantity that the change rate data computing part 224 requires for computation. For example, the data storage part 223 can be configured to store the data of the tremor-frequency feature quantity for one minute. The data storage part 223 can be configured to delete used data after the stress evaluation computing part 225 has completed evaluating the stress, which makes it possible to protect privacy information.

The change rate data computing part (change rate calculation means) 224 calculates a change rate of the tremor-frequency feature quantity with respect to time. In this case, the change rate data computing part 224 can be configured to calculate a change rate using a tremor-frequency feature quantity stored in the data storage part (feature quantity storage means) 223 and a tremor-frequency feature quantity received from the physiological tremor frequency data computing part (tremor feature quantity extracting means) 222.

Specifically, for example, the change rate data computing part 224 receives, from the physiological tremor frequency data computing part 222, the data of three types of feature quantities including the X-coordinate value, the Y-coordinate value, and the total sum of pressure values, and also receives, from the data storage part 223, the previous data for one minute of the three types of the feature quantities. Then the change rate data computing part 224 calculates a change rate from an average value for past one minute of the three types of the feature quantities and an average value for the latest 10 seconds, and sends the change rate data of the three types of the feature quantities to the stress evaluation computing part 225.

The stress evaluation computing part (load evaluation means) 225 evaluates a mental load (stress) of a person exhibiting a physiological tremor (test subject 20) based on the change rate calculated by the change rate data computing part (change rate calculation means) 224. In the above-mentioned example, the stress evaluation computing part 225 receives the change rate data of the three types of feature quantities from the change rate data computing part 224, and calculates an average value of the change rates. If the average value increases, for example, it is assessed that the test subject 20 is under stress. The assessment result is then transmitted through the stress-evaluation-result transmission means 250 to the stress-evaluation-result display (evaluation result display means) 240.

The reason why the average value of the change rates of the three types of the feature quantities is calculated is as follows: The evaluation results having greater accuracy is obtained by evaluating not only a physiological tremor reflected in pressure change but also a physiological tremor reflected in center-of-mass shift in the pressure distribution in the front-back direction and the side-to-side direction of the seat. However, the configuration does not apply only to this. The stress evaluation computing part 225 may be configured to evaluate a mental load (stress) by using a change rate in any one of the feature quantities of an X-coordinate value, a Y-coordinate value, and a total pressure value, for example.

The stress-evaluation-result display (evaluation result display means) 240 displays the evaluation results of a mental load (stress) evaluated by the stress evaluation computing part (load evaluation means) 225.

Next, the evaluation results of the stress using the stress assessment system 200 according to the present example embodiment will be described.

FIG. 4 and FIG. 5 illustrate the evaluation results. Each vertical axis shows body movement of a body trunk (waist portion) that is expressed in acceleration, and indicates an average per hertz (Hz) in the frequency distribution. The horizontal axis shows frequency. In the present example embodiment, the evaluation is performed twice on each of 18 test subjects. In FIG. 4 and FIG. 5, the solid line indicates the results obtained from a test subject given a stress stimulus. The broken line indicates the results obtained from a test subject in a relaxed state.

FIG. 4 illustrates the results of Stroop test that is used as a standard stress stimulus test. FIG. 5 illustrates the results obtained from a test subject who is set a task (an information task) of answering a question about a presented sentence with a time limit being set, which has been developed for the present example embodiment.

In both evaluation results illustrated in FIG. 4 and FIG. 5, there is a marked difference in vibration near 10 hertz (Hz) band between a test subject given a stress stimulus and a test subject in a relaxed state. The difference in frequency band ranging from 8 hertz (Hz) to 10 hertz (Hz) is approximately 0.2 gal in the Stroop test illustrated in FIG. 4 and approximately 0.15 gal with the information task illustrated in FIG. 5. The vibration in the frequency band is caused by a physiological tremor as mentioned above.

Because it is only necessary, in the evaluation of stress using the stress assessment system 200 according to the present example embodiment, to measure body movement directly at a low frequency, the time required to evaluate the stress is only about 10 seconds.

As described above, the stress assessment system 200 according to the present example embodiment makes it possible to measure a physiological tremor of a body trunk instead of a terminal of a body using the pressure distribution sensor 210 with flexible sheet-like structure, for example. In this case, the pressure distribution sensor 210 is closely attached to a body of a test subject on the seat of a chair and the like by weight of the test subject. As a result, the stress assessment system 200 in the present example embodiment allows a simple and quick assessment of the stress without placing stresses on the test subject at all that are caused by wearing a sensor.

Next, a method of assessing stress according to the present example embodiment will be described.

In the method of assessing stress of the present example embodiment, first, pressures on a plurality of places in a plane are detected, and pressure information of information on pressure distribution in the plane is generated. A feature quantity is extracted from the pressure information. Then a tremor-frequency feature quantity is extracted that is a frequency component of the feature quantity and includes a frequency component corresponding to a physiological tremor. Then a change rate of the tremor-frequency feature quantity with respect to time is calculated. Finally, a mental load of a person exhibiting the physiological tremor is evaluated based on the change rate.

The above-mentioned feature quantity can include at least one of central coordinates and a total sum of the pressures in the pressure distribution. The above-mentioned tremor-frequency feature quantity can include a frequency component at 10 hertz of the feature quantity.

As described above, according to the stress assessment system 200 and the method of assessing stress of the present invention, a mental load can be appropriately evaluated without placing a mental load on a test subject.

While the invention has been particularly shown and described with reference to the example embodiments thereof, the invention is not limited to these embodiments. It will be understood by those of ordinary skill in the art that various changes in form and details may be made therein without departing from the spirit and scope of the present invention as defined by the claims.

This application is based upon and claims the benefit of priority from Japanese patent application No. 2016-114380, filed on Jun. 8, 2016, the disclosure of which is incorporated herein in its entirety by reference.

### REFERENCE SIGNS LIST

100 Tremor detector
110 Pressure distribution detecting means
120 Feature quantity extracting means
130 Tremor feature quantity extracting means
200 Stress assessment system
210 Pressure distribution sensor
220 Stress evaluation apparatus
221 Feature quantity data computing part
222 Physiological tremor frequency data computing part
223 Data storage part
224 Change rate data computing part
225 Stress evaluation computing part
230 Pressure data transmission means
240 Stress-evaluation-result display
250 Stress-evaluation-result transmission means
20 Test subject
21 Chair
22 Desk

1. A tremor detector, comprising:
a pressure distribution detecting section configured to detect pressures on a plurality of places in a plane and generate pressure information of information on pressure distribution in the plane;
a feature quantity extracting section configured to extract a feature quantity from the pressure information; and
a tremor feature quantity extracting section configured to extract a tremor-frequency feature quantity that is a frequency component of the feature quantity and includes a frequency component corresponding to a physiological tremor.

2. The tremor detector according to claim 1, wherein
the feature quantity includes at least one of central coordinates and a total sum of the pressures in the pressure distribution.

3. The tremor detector according to claim 1, wherein
the tremor-frequency feature quantity includes the frequency component at 10 hertz of the feature quantity.

4. The tremor detector according to claim 1, wherein
the pressure distribution detecting section has resolution for detecting an amplitude oscillating with acceleration of approximately 0.1 gal and frequency of approximately 10 hertz.

5. The tremor detector according to claim 1, wherein
the pressure distribution detecting section has at least 0.25-micrometer spatial resolution and at least 0.08-pascal pressure resolution, and operates at a sampling rate of at least 20 hertz.

6. A stress assessment system, comprising:
a tremor detector that includes
a pressure distribution detecting section configured to detect pressures on a plurality of places in a plane and generate pressure information of information on pressure distribution in the plane,
a feature quantity extracting section configured to extract a feature quantity from the pressure information, and
a tremor feature quantity extracting section configured to extract a tremor-frequency feature quantity that is a frequency component of the feature quantity and includes a frequency component corresponding to a physiological tremor;
a change rate calculation section configured to calculate a change rate of the tremor-frequency feature quantity with respect to time; and
a load evaluation section configured to evaluate a mental load of a person exhibiting the physiological tremor based on the change rate.

7. The stress assessment system according to claim 6, further comprising:
an evaluation result display section configured to display evaluation results of the mental load evaluated by the load evaluation section; and
a feature quantity storage section configured to store the tremor-frequency feature quantity, wherein
the change rate calculation section calculates the change rate using the tremor-frequency feature quantity stored in the feature quantity storage section and the tremor-frequency feature quantity received from the tremor feature quantity extracting section.

8. The stress assessment system according to claim 6, wherein
the pressure distribution detecting section is configured to be placed on at least one of a seat and a back of a chair for the person exhibiting the physiological tremor to sit on, or under the person who sits on the chair.

9. A method of assessing stress, comprising:
detecting pressures on a plurality of places in a plane, and generating pressure information of information on pressure distribution in the plane;
extracting a feature quantity from the pressure information;
extracting a tremor-frequency feature quantity that is a frequency component of the feature quantity and includes a frequency component corresponding to a physiological tremor;
calculating a change rate of the tremor-frequency feature quantity with respect to time; and
evaluating a mental load of a person exhibiting the physiological tremor based on the change rate.

10. The method of assessing stress according to claim 9, wherein
the feature quantity includes at least one of central coordinates and a total sum of the pressures in the pressure distribution, and
the tremor-frequency feature quantity includes the frequency component at 10 hertz of the feature quantity.

11. The tremor detector according to claim 2, wherein
the tremor-frequency feature quantity includes the frequency component at 10 hertz of the feature quantity.

12. The tremor detector according to claim 2, wherein
the pressure distribution detecting section has resolution for detecting an amplitude oscillating with acceleration of approximately 0.1 gal and frequency of approximately 10 hertz.

13. The tremor detector according to claim 3, wherein
the pressure distribution detecting section has resolution for detecting an amplitude oscillating with acceleration of approximately 0.1 gal and frequency of approximately 10 hertz.

14. The tremor detector according to claim 2, wherein
the pressure distribution detecting section has at least 0.25-micrometer spatial resolution and at least 0.08-pascal pressure resolution, and operates at a sampling rate of at least 20 hertz.

15. The tremor detector according to claim 3, wherein
the pressure distribution detecting section has at least 0.25-micrometer spatial resolution and at least 0.08-pascal pressure resolution, and operates at a sampling rate of at least 20 hertz.

16. The tremor detector according to claim 4, wherein
the pressure distribution detecting section has at least 0.25-micrometer spatial resolution and at least 0.08-pascal pressure resolution, and operates at a sampling rate of at least 20 hertz.

17. The stress assessment system according to claim 6, wherein
the feature quantity includes at least one of central coordinates and a total sum of the pressures in the pressure distribution.

18. The stress assessment system according to claim 6, wherein
the tremor-frequency feature quantity includes the frequency component at 10 hertz of the feature quantity.

19. The stress assessment system according to claim 6, wherein
the pressure distribution detecting section has resolution for detecting an amplitude oscillating with acceleration of approximately 0.1 gal and frequency of approximately 10 hertz.

20. The stress assessment system according to claim 6, wherein
the pressure distribution detecting section has at least 0.25-micrometer spatial resolution and at least 0.08-pascal pressure resolution, and operates at a sampling rate of at least 20 hertz.
